(19) United States
(12) Patent Application Publication
(10) Pub. No.: US 2015/0250592 A1
(43) Pub. Date: Sep. 10, 2015

(54) Methods and Devices for Regenerating a Bone

(71) Applicant: CELGEN AG, Zug (CH)

(72) Inventor: Domonkos HORVATH, Jestetten (DE)

(21) Appl. No.: 14/719,938

(22) Filed: May 22, 2015

Related U.S. Application Data
(63) Continuation of application No. PCT/EP2013/074447, filed on Nov. 22, 2013.
(60) Provisional application No. 61/729,414, filed on Nov. 22, 2012.

(51) Int. Cl. A61F 2/28; A61C 8/02

(52) U.S. Cl. CPC A61F 2/2846; A61C 8/0006

(57) ABSTRACT

Devices for regenerating a bone are provided. The devices include a non-dimensionally stable membrane that is stretchable and that has at least one edge or frame, at least three bone fixing elements provided at the at least one edge or frame, and a hole at a central area of the membrane with a strengthened edge. In addition, methods are provided for regenerating a bone by distraction, or by forwarding and transferring biomechanical impulses into a bone defect.

### CROSS REFERENCE TO RELATED APPLICATIONS

This application is a continuation of International Application No. PCT/EP2013/074447, filed on Nov. 22, 2013, which claims the benefit of U.S. Provisional Application No. 61/729,414, filed on Nov. 22, 2012, the entire contents of which are hereby incorporated by reference.

### FIELD OF THE INVENTION

The present invention relates to methods and devices for regenerating a bone, in particular by distraction, especially by two-dimensional distraction, and to the forwarding and transfer of biomechanical impulses into a bone defect.

### BACKGROUND

Bone losses are generally filled using bone replacement materials, or autogenic or allogenic bone.

Examples of bone replacement materials include inorganic materials such as calcium phosphate, hydroxyapatite, or bioglass, which are replaced by bone after a long absorption period. However, this procedure may be used only for minor defects; otherwise, there is the risk of infection due to insufficient vascularization. Such bone materials do not emit biomechanical pulses and therefore do not initiate active regeneration. There are also synthetically manufactured bone replacement materials made of organic materials, such as polyesters, polyamino acids, polyanhydrides, polyorthoesters, polyphosphazenes, polylactides, or polyglycolides, or made of allogenic organic materials, for example of bovine origin. However, bone substance losses may also be compensated for using microvascular connected autogenic or allogenenically vascularized transplants.

From a biological standpoint, the best replacement material for bone is an autologous spongiosa transplant. However, such transplants have limited availability and exhibit a high absorption rate after transplantation.

The materials and techniques used in the prior art frequently provide unsatisfactory bone quality, resulting, for example, in insecure anchoring of implant beds. In addition, frequently the bone replacement is insufficiently vascularized, thereby increasing the risk of infection. Furthermore, methods of the prior art often use growth factors which greatly increase the costs for the methods.

Bone replacement materials are frequently used in the form of a granulated material, in particular in the mouth and jaw region. Such a granulated material is described in WO 2006/010507 A2, for example. Examples of granulated material known on the market include Bio-Oss® from Geistlich Pharma AG, BONITmatrix® from DOT GmbH, and cyclOS® and Ceros® from Mathys AG.

Bone defects are sometimes covered by a membrane to protect the area of the defect from ingrowing connective tissue. Such membranes are normally just applied in a loose manner.

Instead of using a bone replacement, missing bone substance may sometimes be filled by bone regeneration. Segmented interruptions in the osseous continuity of long tubular bones may be treated in this manner by distraction osteogenesis.

Callus distraction has been known for over a hundred years. The most important biological stimulus for bone formation is mechanical stress. This releases piezoelectric forces which activate the osteoblasts and osteoclasts. Distraction osteogenesis induces new bone formation by triggering biological growth stimuli by means of slow separation of bone segments. This method achieves direct formation of woven bone by distraction. The defined tensile stress is essential for bone formation. When such a defined tensile stress is applied to bone fragments, the mesenchymal tissue exhibits an osteogenetic potential in the gap and at the contiguous fragment ends. When sufficient vascular potency is present, progressive distraction results in metaplasia of the organized hematoma, also referred to as blood coagulum, in a zone of longitudinally arranged fibrous tissue, which under optimal external and internal conditions may be directly converted to woven bone. A complication, however, is that the bone tissue requires highly complex control for regeneration.

WO 01/91663 and U.S. Pat. No. 5,980,252 describe a two-dimensionally oriented bone distraction using a rigid an inelastic artificial interface. For such distraction methods in the prior art, in many cases only vertical regeneration is possible, for example in the jaw region.

Thus, bone regeneration by distraction using a rigid membrane cannot be used for every type of bone defect.

### SUMMARY

A technical problem underlying the present invention is to provide a device which allows bone regeneration methods to be carried out which overcome the disadvantages of the prior art. A further technical object of the invention is the provision of devices which improve the previously known devices for bone regeneration, in particular in a simple manner.

A further technical problem underlying the invention is the provision of devices, use of same, and methods which allow simple and economical bone regeneration.

A further technical object of the invention is the provision of devices, use of same, and methods which allow regeneration of bone and which have improved quality and sufficient vascularization.

In accordance with an example embodiment, a method for enhancing the shape, mass and strength of bone comprises covering a bone defect at least partially with a non dimensionally stable membrane and fixing the frame or edge of the non dimensionally stable membrane to the bone which surrounds the bone defect.

According to an example embodiment, a method for enhancing the shape, mass and strength of bone is provided, which comprises the steps of covering a bone defect at least partially with a non dimensionally stable membrane, fixing the frame or edge of the non-dimensionally stable membrane to the bone which surrounds the bone defect, and moving the non-dimensionally stable membrane away from the bone defect.

This method is called method A.

In a preferred embodiment the membrane is moved with a speed of at least 0.5 mm per day and at most 2.5 mm per day. In a preferred embodiment the membrane is pushed or pulled away from the bone defect.

In a preferred embodiment the membrane is moved away from the bone defect by using a distractor device.

In a preferred embodiment the membrane is moved away from the bone defect by using a screw which is connected to the membrane.

In a preferred embodiment the membrane is moved away from the bone defect until the desired bone structure is formed.

An alternative embodiment of the invention is a method for enhancing the shape, mass and strength of bone, which comprises the steps covering a bone defect at least partially with a non-dimensionally stable membrane, fixing the frame or edge of the non-dimensionally stable membrane to the bone which surrounds the bone defect, filling the interspace of the bone defect between the bone and the non-dimensionally stable membrane up with bone augmentation material such that the non-dimensionally stable membrane is stretched.

This method is called method B.

In a preferred embodiment the non-dimensionally stable membrane is a flexible membrane. In another preferred embodiment the non-dimensionally stable membrane is a stretchable membrane.

The use of a non-dimensionally stable membrane, preferably flexible, preferably stretchable membrane has surprising and new advantages compared with the use of inelastic and rigid membranes according to the state of the art:

Especially when using bone regeneration of the jaw, the space is often very narrow, for example due to adjoining teeth. When using a rigid membrane of the state of the art this is difficult and complex. WO 01/91663 discloses for this purpose for example membranes comprising two pieces of a rigid membrane which have to be bonded together in a very complicating form. Furthermore this solution does not provide a tight connection of the rigid membrane to the tooth.

The use of a non-dimensionally stable membrane, preferably flexible, preferably stretchable membrane according to the invention has the advantage that the membrane can be easier applied in the interspaces of the teeth.

A non-dimensionally stable membrane, preferably flexible, preferably stretchable membrane can surprisingly be used if its frame ore edge is fixed to the bone surrounding the bone defect, for example, in a peripheral circular manner around the bone defect.

Studies with monolayer-cell cultures of osteoblasts on carrier plates show that the bending and the stretching of the carrier plates results in a stretching of the osteoblasts which leads to a cell differentiation similar as during callus distraction.

Method A has the following advantages:

Since the membrane which is fixed to the bone is at the same time moved according to method A, the membrane is preferably flexible and stretchable. This has a further advantage: There is not only a distraction of the cells attaching to the membrane due to the movement of the membrane away from the bone defect as it is known for stiff membranes, but there is also a distraction of the cells attaching to the membrane due to stretching of the membrane. The stretched membrane replaces a stiff membrane according to the state of the art.

The periosteum is like sinews not stretchable. Furthermore, some kind of stretching the periosteum would lead to pains of the patient. Therefore the periosteum can not be used for distraction methods. The stretchable membrane replaces the non-stretchable periosteum. Therefore, method A could also be called artificial periosteum distraction.

Method B has the following advantages:

It was found that biomechanical stimuli and impulses are more important for bone regeneration than other factors. When using the augmentation method, where bone defects are filled with augmentation material, it was generally assumed that a protective barrier between the augmented bone defect and the surrounding connective tissue is more important. As protective barrier, nonporous membranes were used which are not connected to the bone or are at most simply fixed in their position.

Method B works constitutively different. Membranes are used which are preferably porous or perforated, which allow a vascularization and an immigration of cells including fibroblasts into the bone defect filled up with the augmentation material. This results in a good and tight binding of the non-dimensionally stable membrane to the bone resulting further to the effect that the membrane forwards biomechanical stimuli from outside into the bone defect which is filled up with augmentation material so that the membrane is stretched. The biomechanical stimuli and impulses can result, for example, from the movement of the mouth while speaking or eating. Due to the stretched membrane and the augmentation material filled up in the bone defect and contacting the membrane, the cells in the bone defect receive also the biomechanical stimuli and impulses.

In an alternative example embodiment the methods according to the present invention, especially method A and/or method B, are used for the regeneration of the periodont. Since not only the bone is regenerated in a periodontal regeneration but also the cementum of the root, the periodontal fiber and the gingiva, such as regeneration is a specific kind of callus distraction. This kind is technical difficult when using a membrane which is moved. The use of a non-dimensionally stable membrane, preferably flexible, preferably stretchable membrane makes this method easier or even possible at all since the membrane can follow the form of the teeth and can be compressed, e.g. due to wrinkles when it reaches smaller distances between the teeth.

In a preferred example embodiment the non-dimensionally stable membrane is fixed to the bone by at least two fixing points.

In a preferred example embodiment the non-dimensionally stable membrane is fixed to the bone by at least three fixing points.

In a preferred example embodiment the non-dimensionally stable membrane is fixed to the bone by at least four fixing points. In a preferred example embodiment the non-dimensionally stable membrane is fixed to the bone by four, five, six, seven eight, nine or ten fixing points.

In a preferred example embodiment the non-dimensionally stable membrane is fixed to the bone by screws, pins, or nails.

Preferably, the screws, pins, or nails are biodegradable.

In a preferred example embodiment the non-dimensionally stable membrane is fixed to the bone by gluing or fusing the frame or edge of the non-dimensionally stable membrane to the bone.

In a preferred example embodiment the bone defect is surrounded from at least three sides with bone. In a preferred example embodiment the bone defect is surrounded from at least four sides with bone.

In a preferred example embodiment the bone is an alveolar or intramembraneous bone. In a preferred example embodiment the bone is a jaw. In a preferred example embodiment the bone is an upper jaw or a lower jar.

In a preferred example embodiment the bone defect is between two teeth.

In a preferred example embodiment two teeth are neighboring teeth.

In a preferred example embodiment the methods according to the present invention use a device according to the present invention.

In a preferred example embodiment the methods according to the present invention use a membrane according to the present invention.

According to another example embodiment, a device for enhancing the shape, mass and/or strength of bone is provided, comprising a non-dimensionally stable membrane having at least one frame or edge, means for fixing the frame or edge of the non-dimensionally stable membrane to the bone and a distractor or screw for moving, pushing or pulling said non-dimensionally stable membrane.

In a preferred example embodiment the distractor is connected to the membrane. Preferably, the distractor is connected to the central area of the membrane. In an alternative example embodiment the screw is connected to the membrane. Preferably, the screw is connected to the central area of the membrane.

Preferably, the device according to the present invention further comprises means for attaching said distractor to dental structures.

Preferably, the distractor or screw is connected to the membrane via a washer and/or a grommet.

In a preferred example embodiment the membrane of the device according to an example embodiment is a membrane which is described throughout the description. Preferably the membrane is non-dimensionally stable and comprises at least one edge or frame, wherein the membrane comprises at the edge or frame at least three bone-fixing elements, more preferably at least four bone-fixing elements, and wherein the membrane has at the central area of the membrane a hole with a strengthened edge.

Preferably the non-dimensionally stable membrane is a flexible membrane. More preferably, the non-dimensionally stable membrane is a stretchable membrane.

Preferably the membrane has at least one hole. Preferably the edge of the hole in the membrane comprises a grommet.

A person of ordinary skill in the art knows suitable materials for a non-dimensionally stable membrane, especially a flexible membrane or a stretchable membrane. Such materials can be for example elastomers and materials, for example plastic materials, with a high viscosity.

Further preferred example embodiments of the membrane are disclosed throughout the description.

According to an example embodiment, the device comprises means for fixing the frame or edge of the non-dimensionally stable membrane to a bone. Such means for fixing, also called bone-fixing elements, are preferably pins, nails or screws. Preferably, the pins, screws or nails are biodegradable. In a preferred example embodiment, the pins, nails or screws are connected to the membrane via grommets. In a preferred example embodiment, the grommets are bioresorbable, i.e. are made from a bioresorbable material like polylactide or polycaprolactone.

In a preferred example embodiment, the devices are used in a method.

According to a further example embodiment, a device for enhancing the shape, mass and strength of bone is provided comprising a non-dimensionally stable membrane, means for fixing the frame or edge of the non-dimensionally stable membrane to a bone and a distractor for moving, pushing or pulling said non-dimensionally stable membrane.

In a preferred example embodiment, the distractor is connected to the membrane. In a preferred embodiment the distractor is connected to the central area of said membrane.

In a preferred example embodiment, the device further comprises means for attaching said distractor to dental structures.

According to another example embodiment, a device for enhancing the shape, mass and strength of bone is provided comprising a non dimensionally stable membrane, means for fixing the frame or edge of the non dimensionally stable membrane to a bone and a screw for moving, pushing or pulling said non dimensionally stable membrane.

In a preferred example embodiment, the screw is connected to the membrane. In a preferred example embodiment, the screw is connected to the central area of said membrane.

In a preferred example embodiment, the screw is connected to the membrane via a washer and/or a grommet.

A washer between the screw and the membrane has the advantage that the screw can not slip through the membrane but the membrane is hold at the screw at a specific point. The washer can be used as the bearing area of the membrane on the screw.

A grommet surrounding the hole of the membrane in which the screw is placed has the advantage that a small area of the membrane around the hole is stiff, and therefore suitable to be the fixing point between the screw and the stretched membrane.

In a preferred example embodiment the washer and/or the grommet are bioresorbable, i.e. are made from a bioresorbable material like polylactide or polycaprolactone.

In a preferred example embodiment the device comprises a membrane.

According to an example embodiment, a membrane is provided which is non-dimensionally stable, especially which is flexible or stretchable, wherein the membrane comprises at the edge or frame at least three pins, nails or screws, more preferably at least four pins, nails or screws, and wherein the membrane has at the central area of the membrane a hole with a strengthened edge.

According to another example embodiment, a membrane is provided which is non-dimensionally stable, especially which is flexible or stretchable, wherein the membrane comprises at the edge or frame at least three nails, more preferably at least four nails, and wherein the membrane has at the central area of the membrane a hole with a strengthened edge.

According to an example embodiment, a membrane is provided which is non-dimensionally stable, especially which is flexible or stretchable, wherein the membrane comprises at the edge or frame at least three pins, more preferably at least four pins, and wherein the membrane has at the central area of the membrane a hole with a strengthened edge.

According to an example embodiment, a membrane is provided which is non-dimensionally stable, especially which is flexible or stretchable, wherein the membrane comprises at the edge or frame at least three screws, more preferably at least four screws, and wherein the membrane has at the central area of the membrane a hole with a strengthened edge.

In a preferred example embodiment, the edge of the hole comprises a grommet.

In a preferred example embodiment, the membrane is part of a device according to an example embodiment.

In a preferred example embodiment, the membrane is used in a method according to an example embodiment.

Preferred features of the method according to the example embodiment are also disclosed for the devices and membranes according to an example embodiment.

Preferred features of the devices according to an example embodiment are also disclosed for the methods and membranes according to an example embodiment.

Preferred features of the membranes according to an example embodiment are also disclosed for the devices and methods according to an example embodiment.

In a preferred embodiment the membrane is a deformable membrane.

The devices and membranes according to an example embodiment may be advantageously used in methods, preferably methods according to an example embodiment, for bone regeneration, in particular for two-dimensional callus distraction.

In particular, the term “bone regeneration” is also understood to mean the regeneration of bone defects, for example after cystectomy, tumor surgery, or trauma surgery, etc., regardless of the topography, and/or in particular also means the regeneration of minor bone defects, for example those caused by periodontitis.

However, bone outside the jaw region and/or outside the periodontal region may also be regenerated.

It may be provided, for example, that membrane is not biogenic, in particular that the membrane contains no collagen or is collagen-free. However, it may also be provided that the membrane is biogenic. The membrane can contain collagen or can be al collagen based membrane.

The membrane and its parts may be made from one or multiple biodegradable materials.

According to an example embodiment, the membrane and/or the pins, screws or nails and/or the washer and/or the grommet can contain a material selected from the group comprising polyglycolic acid, polylactic acid, poly(ε-caprolactone), poly(β-hydroxybutyrate), poly(p-dioxanone), a polyanhydride, or a mixture of same, for example a mixture of polylactic acid and polyglycolic acid. According to the invention the membrane preferably contains polylactic acid. According to an example embodiment, the membrane preferably contains poly(ε-caprolactone). According to another example embodiment, the membrane preferably contains a carbolactone.

According to an example embodiment the pins, screws or nails are preferably composed of polylactic acid or of poly(ε-caprolactone).

According to an example embodiment, the membrane preferably has at least one cell adhesive property; i.e., it is able to bind cells, in particular osteoblasts, fibroblasts, and/or endothelial cells, and preferably is able to bind specifically and selectively. According to an example embodiment, the cell adhesive property of the membrane is preferably determined by its surface characteristics.

The membrane can be coated with hydroxyapatite. A coating with hydroxyapatite can allow adsorption of proteins, which promotes binding.

The membrane can be coated with at least one protein. According to an example embodiment the at least one protein preferably contains the amino acid sequence Arg-Gly-Asp, i.e., RGD. According to an example embodiment, the membrane is coated with at least one peptide. According to an example embodiment, the at least one peptide is preferably a peptide which initiates the cell adhesion. According to an example embodiment, the at least one peptide is preferably an RGD peptide. According to an example embodiment, the at least one peptide is preferably synthetically produced. According to an example embodiment, the at least one peptide preferably contains the amino acid sequence Arg-Gly-Asp, i.e., RGD. According to an example embodiment, the at least one peptide preferably comprises the amino acid sequence Arg-Gly-Asp, i.e., RGD.

According to an example embodiment, the membrane is coated with star-shaped polyethylene glycol polymers (star PEG).

The adhesion of osteoblasts is a receptor-mediated contact between the molecules of the extracellular matrix and the actin fibers of the cytoskeleton. This region is also referred to as the focal contact zone. Molecules which provide for binding as well as molecules which are responsible for signal transduction are present in the focal contacts. Formation of the focal adhesion is caused primarily by integrins. The integrins differ from other cell surface receptors by virtue of their bio affinity. Adhesion proteins in the form of an ultrathin coating on the membrane facilitate the adhesion binding of osteoblasts to the device according to the invention. Fibronectin is an extracellular adhesion protein having multiple specific binding sites for receptors, and is therefore used for binding the osteoblasts to the extracellular matrix. Fibronectin is a large glycoprotein, which as a dimer is composed of two essentially identical subunits. Fibronectin is composed of approximately 90 amino acids. The cell-binding site of fibronectin has been identified as the tripeptide sequence Arg-Gly-Asp (RGD).

The surface of the membrane can be chemically modified.

According to an example embodiment, the membrane is preferably permeable to a liquid. According to an example embodiment, the membrane is preferably permeable to water. According to an example embodiment, the membrane is preferably porous. According to an example embodiment, the membrane preferably has pores which are permeable to water and to solids, for example proteins and sugars having a mass of less than 100 kDa, particularly preferably less than 50 kDa. According to an example embodiment, the membrane preferably has pores which are non-permeable to solids, for example proteins and sugars having a mass of greater than 50 kDa, particularly preferably greater than 100 kDa, in particular greater than 150 kDa. According to an example embodiment, the pores preferably have a size of 2 μm maximum, particularly preferably 1 μm maximum. According to an example embodiment, the pores preferably have a size of 0.5 μm maximum, particularly preferably 0.1 μm maximum. According to an example embodiment, the pores preferably have a size of at least 0.01 μm, particularly preferably at least 0.05 μm. According to an example embodiment, the pores preferably have a size of at least 0.1 μm, particularly preferably at least 0.5 μm. According to an example embodiment, the pores preferably have a size of 1 μm.

According to an example embodiment, the pores preferably have a size of 5 mm maximum, particularly preferably 2 mm maximum. According to an example embodiment, the pores preferably have a size of 1 mm maximum, particularly preferably 0.5 mm maximum.

According to an example embodiment, the membrane preferably has a plurality of pores, e.g. 2 pores, 3 pores, 4, pores, 5 pores, 6 pores, 7 pores, 8 pores, 9 pores 10 pores or more, for example 10 to 100 pores.

According to an example embodiment, the membrane is preferably biocompatible. According to an example embodiment, the membrane is preferably biodegradable.

In the context of the present invention, “biodegradable” is understood to mean that the material may be degraded or absorbed by hydrolysis, polymer degradation, enzymatic decomposition, and/or dissociation of the material components, preferably in an organism, for example a human or animal organism. According to the invention, the degradation products of the particles preferably have a molecular weight of 50,000 g/mol maximum, particularly preferably 40,000 g/mol maximum. Thus, they may be excreted in the normal manner.

According to an example embodiment, the biodegradable, deformable membranes are preferably degraded in an organism within an absorption time of two years, particularly preferably within one year, in particular within one month, most preferably within two weeks.

In one alternative example embodiment, the deformable, in particular expandable, membranes are porous. In one alternative example embodiment, the deformable, in particular expandable, membranes are nonporous.

According to an example embodiment, the membrane preferably transmits biomechanical pulses, in particular expansion stimuli or pressure stimuli, to the cells surrounding the granulate mixture, so that the cells may be distracted or compressed by distances of at least 0.5 μm, in particular 1 μm, more preferably 2 μm, most preferably 10 μm to preferably 100 μm, very particularly preferably 1000 μm, more particularly preferably 1 cm, most particularly preferably up to 10 cm. Thus, according to the invention the

However, cells surrounding the augmentation material may also experience a pressure pulse as a result of the pressure to the membrane. The pulses may also be transferred via the body's own fibrin network. However, in particular the pulses are relayed to the cells also via the non-deformable particles of the augmentation material.

According to an example embodiment, the biomechanical pulses are preferably transmitted at a maximum distraction rate of 0.5 to 2.5 mm/day. According to an example embodiment, the biomechanical pulses are preferably transmitted at a maximum distraction rate of 1 mm/day. According to an example embodiment, the expansion stimuli are preferably transmitted at a maximum distraction rate of around 1 mm/day. According to the invention, the pressure stimuli are preferably transmitted at a maximum distraction rate of around 1 mm/day.

In one example embodiment the augmentation material comprises or consists of particles, e.g. non-deformable particles.

In one example embodiment, it may be provided in particular that the augmentation material contains a bone replacement material.

In one example embodiment, it may be provided in particular that the bone replacement material is an organic or an inorganic bone replacement material.

In one example embodiment, it may be provided in particular that the bone replacement material is allogenic or autogenic bone.

In one example embodiment, it may be provided in particular that the augmentation material contains hydroxyapatite and/or tricalcium phosphate.

Suitable augmentation material according to the state of the art is known by a person skilled in the art.

According to an example embodiment, the augmentation material is preferably produced in vitro.

In one alternative example embodiment, the augmentation material is material known on the market, such as Bio-Oss® from Geistlich Pharma AG, BONITmatrix® from DOT GmbH, or cyclOS® and Ceros® from Mathys AG.

According to an example embodiment, a kit, i.e. a kit of parts is provided comprising a membrane according to an example embodiment and augmentation material. Preferably the augmentation material is augmentation material disclosed throughout the present description.

Preferably the kit comprises an instruction manual. Preferably the instruction manual discloses the use of the kit in method B.

According to a further example embodiment, a method for regenerating a bone is provided, wherein a device or membrane according to an example embodiment is introduced into a defect region of a bone.

According to an example embodiment, medical procedures are provided in which a device or membrane according to an example embodiment is used.

According to another example embodiment, the first medical indication of a device or membrane is provided according to an example embodiment.

In one embodiment according to the invention, the bone defect is revivified before the device or membrane is introduced.

In one example embodiment, the distraction takes place over a period of at least one day, in particular at least 2 days, and a maximum of 300 days, in particular a maximum of 100 days.

In one example embodiment, the distraction takes place over a period of at least one day. In one example embodiment, the distraction takes place over a period of at least 2 days. In one example embodiment, the distraction takes place over a period of at least 5 days. In one example embodiment, the distraction takes place over a period of at least 10 days.

In one example embodiment, the distraction takes place over a maximum period of 300 days. In one example embodiment, the distraction takes place over a maximum period of 100 days. In one example embodiment, the distraction takes place over a maximum period of 50 days.

In one example embodiment, the distraction takes place over a period of several days, in particular over a period of 5 to 20 days, particularly preferably over a period of approximately 10 days, in particular 10 days.

In conjunction with an example embodiment, cell distraction is understood to mean the distraction of individual cells, in particular osteoblasts. These individual cells attach to the membrane or the augmentation material, and experience direct or indirect distraction pulses as a result of the stretching and/or deformation of the deformable membrane. In one example embodiment, a distraction pulse experienced by a cell, in particular an osteoblast, is 1 μm to 10 μm. In one example embodiment, the distraction distance a cell, in particular an osteoblast, is pulled is 1 μm to 200 μm. In one example embodiment, the distraction distance a cell, in particular an osteoblast, is pulled is at least 1 μm to a maximum of 10 μm. In one example embodiment, the distraction distance a cell, in particular an osteoblast, is pulled is at least 10 μm to a maximum of 200 μm.

In one example embodiment, the rate at which a cell, in particular an osteoblast, is pulled is at least 1 μm/day.

In conjunction with the present invention, tissue distraction is understood to mean the distraction of a tissue, for example a bone tissue, in particular a callus. The tissue is thus composed of a plurality of cells, in particular also osteoblasts. The tissue, in particular a callus, attaches to the deformable or non-deformable particles and experiences direct or indirect distraction pulses as a result of the movement and stretching/deformation of the membrane. In one example embodiment, a distraction pulse experienced by a tissue, in particular a callus, is 1 μm to 1000 μm. In one example embodiment, the distraction distance a tissue, in particular a callus, is pulled is 10 μm to 30 cm. In one example embodiment, the distraction distance a tissue, in particular a callus, is pulled is 10 μm to 3 cm. In one example embodiment, the distraction distance a tissue, in particular a callus, is pulled is 10 μm to 10 mm. In one example embodiment, the distraction distance a tissue, in particular a callus, is pulled is at least 0.2 mm to a maximum of 5 mm.

In one example embodiment, the distraction distance a tissue, in particular a callus, is pulled is at least 10 μm. In one example embodiment, the distraction distance a tissue, in particular a callus, is pulled is at least 100 μm. In one example embodiment, the distraction distance a tissue, in particular a callus, is pulled is at least 1 mm. In one example embodiment, the distraction distance a tissue, in particular a callus, is pulled is 30 cm maximum. In one example embodiment, the distraction distance a tissue, in particular a callus, is pulled is 10 cm maximum. In one example embodiment, the distraction distance a tissue, in particular a callus, is pulled is 3 cm maximum. In one example embodiment, the distraction distance a tissue, in particular a callus, is pulled is 1 cm maximum. In one example embodiment, the distraction distance a tissue, in particular a callus, is pulled is 0.5 cm maximum.

In one example embodiment, the rate at which a tissue, in particular a callus, is pulled is at least 10 μm/day. In one example embodiment, the rate at which a tissue, in particular a callus, is pulled is at least 0.1 mm/day. In one example embodiment, the rate at which a tissue, in particular a callus, is pulled is at least 0.25 mm/day. In one example embodiment, the rate at which a tissue, in particular a callus, is pulled is 2.5 mm/day maximum. In one example embodiment, the rate at which a tissue, in particular a callus, is pulled is approximately 1 mm/day.

In one example embodiment, the rate at which a tissue, in particular a callus, is pulled is at least 0.25 mm/day and a maximum of 2 mm/day. In one example embodiment, the rate at which a tissue, in particular a callus, is pulled is at least 0.5 mm/day and a maximum of 2 mm/day. In one example embodiment, the rate at which a tissue, in particular a callus, is pulled is at least 0.5 mm/day and a maximum of 1.5 mm/day.

The method according to an example embodiment uses the body's own healing mechanisms as a bioreactor. Thus, the bone formation occurs under natural conditions, so that the necessary aspects such as growth factors, hormones, and cell composition are implicitly taken into account. In this manner the method according to an example embodiment overcomes problems which may arise as a result of the highly complex control for bone regeneration, as well as the problems of a slow and complicated bone regeneration process using distraction methods from the prior art.

According to an example embodiment, the bone defect is preferably revivified before the device or membrane according to the invention is introduced. According to an example embodiment, in the method according to an example embodiment before the device or membrane according to an example embodiment is introduced into a bone defect this defect is preferably surgically revivified, and in particular bleeding is induced. A blood clot forms in the defect as a result of the surgical revivification and the induced bleeding.

According to another example embodiment, a second medical indication of devices and membranes is provided, for regenerating a bone, in particular a bone in the jaw region.

According to an example embodiment, a kit for bone regeneration is provided, containing a membrane according to an example embodiment and screws, pins or nails.

Further devices such as a surgical instrument, an instruction manual, and/or a package may be associated with the membrane or device or kit according to an example embodiment.

### BRIEF DESCRIPTION OF THE DRAWINGS

Example embodiments will now be described with reference to the drawings.

FIG. 1A shows a bone with a bone defect to be treated according to an example embodiment.

FIG. 1B shows a bone defect and a device for regenerating the bone according to an example embodiment.

FIG. 1C shows a bone defect and a device for regenerating the bone in a upward position according to an example embodiment.

FIG. 2A shows a bone with teeth to be treated according to method A according to an example embodiment.

FIG. 2B shows a flexible membrane according to an example embodiment.

FIG. 2C is another view of the flexible membrane according to an example embodiment.

FIG. 3A shows a bone with a bone defect to be treated according to an example embodiment.

FIG. 3B shows a bone defect filled up with an augmentation material according to an example embodiment.

FIG. 3C shows a membrane fixed to the bone and stretched by an augmentation material according to an example embodiment.

FIG. 3D is a cross section view of a membrane fixed to the bone and stretched by an augmentation material according to an example embodiment.

### DETAILED DESCRIPTION

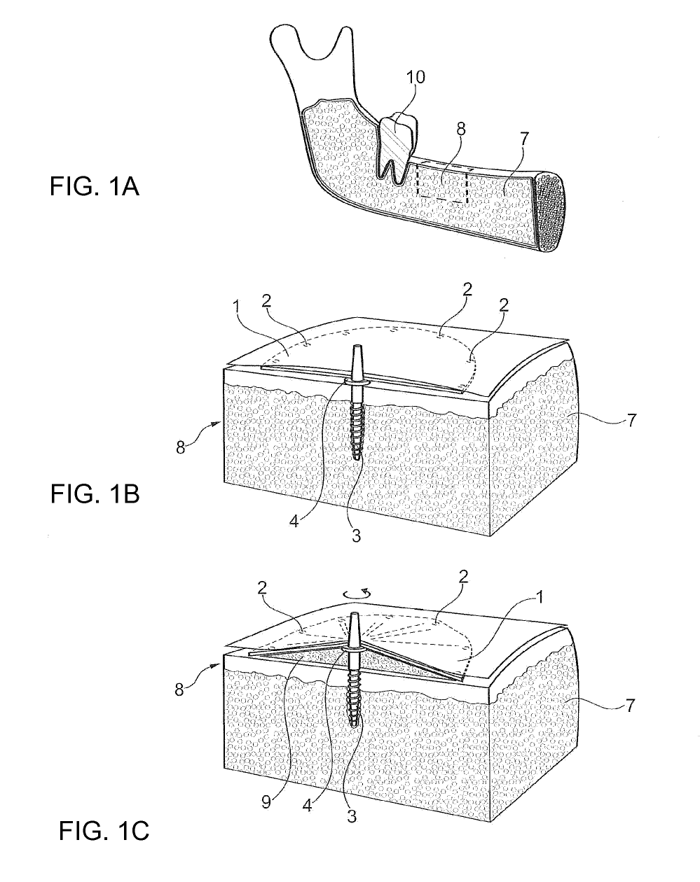

FIGS. 1A-1C schematically show method A and a device according to an example embodiment. FIG. 1A shows a jaw bone 7 with a tooth 10 and a site of a bone defect 8. FIG. 1B shows the bone defect 8 with a device according to an example embodiment having a flexible membrane 1 fixed to the surrounding bone 7 with pins 2 and a screw 3 penetrating the membrane 1 in the middle area. A washer 4 of the screw 3 holds the membrane 1. As shown in FIG. 1C the screw 3 can be screwed upwards so that the membrane 1 is stretched leading to a distraction as outlined above. Preferably the membrane 1 is moved away from the bone defect 8 with a speed of around 0.5 to 2.5 mm per day.

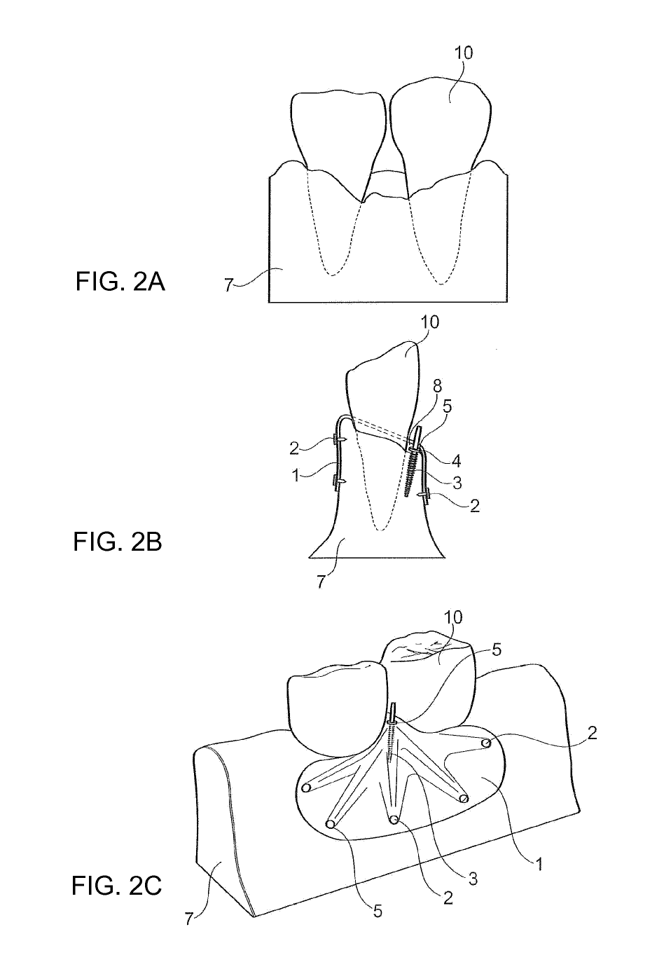

FIGS. 2A-2C schematically show method A and a device according to another example embodiment when used in the interspace between two adjacent teeth 10. As outlined above a flexible membrane 1 can be us advantageously in such interspaces. The membrane 1 of the device has a grommet 5 to better fix the screw 3 and the washer 4 and further grommets 5 for the pins 2. As can be seen from FIG. 2B the screw 3 has not to penetrate the membrane 1 exactly in the middle but can penetrate the membrane 1 at a suitable area.

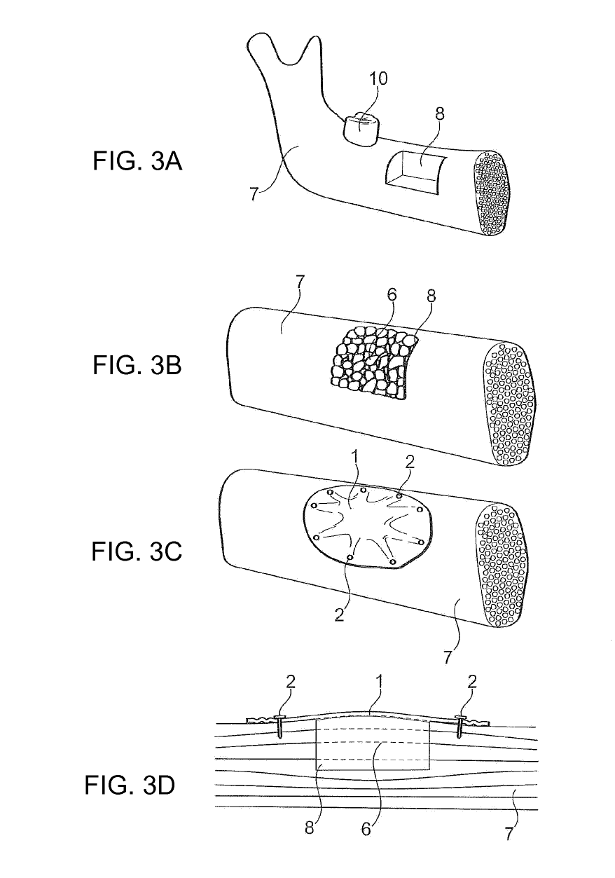

FIGS. 3A-3D schematically show method B. FIG. 3A shows a jaw bone 7 with a tooth 10 and a site of a bone defect 8. FIG. 3B shows the bone defect 8 filled up with an augmentation material 6. FIG. 3C shows the membrane 1 fixed to the bone 7 and stretched by the augmentation material 6 in the bone defect. FIG. 3D shows an according cross section. The horizontal stripes symbolize the forwarding of biomechanical impulses from outside to the bone 7. At the site of the bone defect 8 these biomechanical impulses are forwarded by the stretched membrane 1 to the augmentation material 6 and accordingly to the cells at the bone defect 8 resulting in bone regeneration.

In summary, a device for enhancing the shape, mass and strength of bone is provided, comprising: a non dimensionally stable membrane having at least one frame or edge, wherein the membrane is stretchable, means for fixing the frame or edge of the non dimensionally stable membrane to a bone, a distractor or a screw for moving, pushing or pulling said non dimensionally stable membrane. The distractor or screw is connected to the membrane.

According to an example embodiment, the distractor or screw is connected to the central area of said membrane.

According to another example embodiment, the device further comprises means for attaching said distractor to dental structures. The distractor or screw is connected to the membrane via a washer and/or a grommet.

According to a further example embodiment, the device comprises a membrane according to the following example embodiments.

According to an example embodiment, the non-dimensionally stable membrane is an expandable membrane.

According to another example embodiment, distractor or the screw is for pushing or pulling said non-dimensionally stable membrane.

According to a further example embodiment, a membrane is provided which is non-dimensionally stable comprising at least one edge or frame, wherein the membrane comprises at the edge or frame at least three bone fixing elements, more preferably at least four bone fixing elements, and wherein the membrane has at the central area of the membrane a hole with a strengthened edge, wherein the membrane is stretchable.

According to yet another example embodiment, the bone fixing elements are pins, nails or screws.

According to an example embodiment, the edge of the hole comprises a grommet.

According to another example embodiment, a kit is provided comprising a membrane according to one of the example embodiments and augmenting material.

According to a further example embodiment, a method for enhancing the shape, mass and strength of bone is provided, which comprises: covering a bone defect at least partially with a non dimensionally stable membrane, fixing the frame or edge of the non dimensionally stable membrane to the bone which surrounds the bone defect.

According to yet another example embodiment, a method for enhancing the shape, mass and strength of bone is provided, which comprises covering a bone defect at least partially with a non dimensionally stable membrane, fixing the frame or edge of the non dimensionally stable membrane to the bone which surrounds the bone defect; and moving said non-dimensionally stable membrane away from the bone defect.

According to an example embodiment, a method for enhancing the shape, mass and strength of bone is provided, which comprises covering a bone defect at least partially with a non dimensionally stable membrane, fixing the frame or edge of the non dimensionally stable membrane to the bone which surrounds the bone defect, and filling the interspace of the bone defect between the bone and the non dimensionally stable membrane up with bone augmentation material so that the non dimensionally stable membrane is stretched.

According to another example embodiment, the non-dimensionally stable membrane is a flexible membrane or a stretchable membrane. The non-dimensionally stable membrane is fixed to the bone by at least two fixing points, more preferably at least three fixing points, most preferably at least four fixing points.

According to a further example embodiment, the non-dimensionally stable membrane is fixed to the bone by pins, screws or nails. The non-dimensionally stable membrane is fixed to the bone by gluing or fusing the frame or edge of the non dimensionally stable membrane to the bone. The bone defect is surrounded from at least three sides with bone. The membrane is moved with a speed of at least 0.5 mm per day and at most 2.5 mm per day.

According to yet another example embodiment, the membrane is moved away from the bone defect by using a distractor device or by using a screw which is connected to the membrane.

According to another example embodiment, the bone defect is between two teeth.

What is claimed is:

1. A device for enhancing the shape, mass and strength of a bone, comprising:
a non-dimensionally stable membrane having at least one frame or edge, wherein the membrane is stretchable;
means for fixing the frame or edge of the non-dimensionally stable membrane to the bone; and
a distractor or a screw configured to move, push or pull the non-dimensionally stable membrane.

2. The device according to claim 1, wherein the distractor or the screw is connected to the non-dimensionally stable membrane.

3. The device according to claim 2, wherein the distractor or the screw is connected to a central area of the non-dimensionally stable membrane.

4. The device according to claim 1, further comprising means for attaching the distractor to dental structures.

5. The device according to claim 1, wherein the distractor or the screw is connected to the non-dimensionally stable membrane via a washer and/or a grommet.

6. The device according to claim 1, wherein the non-dimensionally stable membrane comprises:
at least one edge or frame;
at least three bone fixing elements provided at the at least one edge or frame; and
a hole at a central area of the membrane with a strengthened edge,
wherein the non-dimensionally stable membrane is stretchable.

7. The device according to claim 1, wherein the non-dimensionally stable membrane is an expandable membrane.

8. The device according to claim 1, wherein the distractor or the screw is configured to push or pull the non-dimensionally stable membrane.

9. A non-dimensionally stable membrane comprising:
at least one edge or frame;
at least three bone fixing elements provided at the at least one edge or frame; and
a hole at a central area of the membrane with a strengthened edge,
wherein the non-dimensionally stable membrane is stretchable.

10. The membrane according to claim 9, wherein the at least three bone fixing elements comprise pins, nails or screws.

11. The membrane according to claim 9, wherein the edge of the hole comprises a grommet.

12. A kit comprising:
a membrane according to claim 9, and
augmenting material.

13. A method for enhancing the shape, mass and strength of bone, comprising:
covering a bone defect at least partially with a non-dimensionally stable membrane;
fixing a frame or an edge of the non-dimensionally stable membrane to the bone which surrounds the bone defect.

14. A method for enhancing the shape, mass and strength of bone, comprising:
covering a bone defect at least partially with a non-dimensionally stable membrane;
fixing the frame or edge of the non-dimensionally stable membrane to the bone which surrounds the bone defect; and
moving the non-dimensionally stable membrane away from the bone defect.

15. A method for enhancing the shape, mass and strength of bone, comprising:
covering a bone defect at least partially with a non-dimensionally stable membrane;
fixing the frame or edge of the non-dimensionally stable membrane to the bone which surrounds the bone defect;
filling the interspace of the bone defect between the bone and the non-dimensionally stable membrane up with bone augmentation material such that the non-dimensionally stable membrane is stretched.

16. The method according to claim 13, wherein the non-dimensionally stable membrane is a flexible membrane or a stretchable membrane.

17. The method according to claim 13, wherein the non-dimensionally stable membrane is fixed to the bone by at least two fixing points.

18. The method according to claim 13, wherein the non-dimensionally stable membrane is fixed to the bone by pins, screws or nails.

19. The method according to claim 13, wherein the non-dimensionally stable membrane is fixed to the bone by gluing or fusing the frame or edge of the non-dimensionally stable membrane to the bone.

20. The method according to claim 13, wherein the bone defect is surrounded by at least three sides with the bone.

21. The method according to claim 14, wherein the membrane is moved with a speed in the range between at least 0.5 mm per day and at most 2.5 mm per day.

22. The method according to claim 14, wherein the membrane is moved away from the bone defect by using a distractor device or by using a screw which is connected to the non-dimensionally stable membrane.

23. The method according to claim 13, wherein the bone defect is between two teeth.
